(19) United States
(12) Patent Application Publication
(10) Pub. No.: US 2014/0121538 A1
(43) Pub. Date: May 1, 2014

(54) NEEDLE WITH AN OPTICAL FIBER INTEGRATED IN AN ELONGATED INSERT

(71) Applicants: Bernardus Hendrikus Wilhelmus Hendriks, Eindhoven (NL); Waltherus Cornelis Jozef Bierhoff, Veldhoven (NL); Gerhardus Wilhelmus Lucassen, Eindhoven (NL); Jeroen Jan Lambertus Horiks, Weert (NL); Susanne Dorien Van Den Bergdams, Eindhoven (NL); Christian Reich, Eindhoven (NL); Stephan Voss, Schwerin (DE); Axel Winkel, Zapel-Hof (DE); Rami Nachabe, Eindhoven (NL); Manfred Muller, Eindhoven (NL); Marjolein Van Der Voort, Valkenswaard (NL)

(72) Inventors: Bernardus Hendrikus Wilhelmus Hendriks, Eindhoven (NL); Waltherus Cornelis Jozef Bierhoff, Veldhoven (NL); Gerhardus Wilhelmus Lucassen, Eindhoven (NL); Jeroen Jan Lambertus Horiks, Weert (NL); Susanne Dorien Van Den Bergdams, Eindhoven (NL); Christian Reich, Eindhoven (NL); Stephan Voss, Schwerin (DE); Axel Winkel, Zapel-Hof (DE); Rami Nachabe, Eindhoven (NL); Manfred Muller, Eindhoven (NL); Marjolein Van Der Voort, Valkenswaard (NL)

(73) Assignee: KONINKLIJKE PHILIPS N.V., EINDHOVEN (NL)

(21) Appl. No.: 14/127,692

(22) PCT Filed: Jun. 13, 2012
(86) PCT No.: PCT/IB2012/052978

§ 371 Date: Dec. 19, 2013

(30) Foreign Application Priority Data
Jun. 28, 2011 (EP) 11171667.6

(51) Int. Cl. A61B 5/00; A61B 10/02; A61B 1/00; A61B 1/06; A61B 1/07

(52) U.S. Cl. CPC A61B 5/6848; A61B 5/0084; A61B 1/0684; A61B 1/0646; A61B 1/07; A61B 1/00009; A61B 5/0073; A61B 5/0075; A61B 10/0233
USPC 600/478; 29/428

(57) ABSTRACT

A needle is proposed including a cannula or hollow shaft with a multilumen insert inside. The insert comprises at least two lumen. Both the insert as well as the cannula have bevelled ends. In the insert substantially straight cleaved fibers are present that may be connected at the proximal end to a console. At least one of the distal fiber ends in the insert may protrude more than half the fiber diameter out of the insert. Furthermore, the bevel angle of the insert is different from the bevel angle of the cannula such that combination cannula and insert is such that the fiber ends do not protrude the bevel surface of the cannula.

### FIELD OF THE INVENTION

The invention generally relates to a needle with integrated fibers. Particularly, the invention relates to a system including a small diameter needle for tissue inspection based on diffuse reflectance and autofluorescence measurements to diagnose whether tissue is cancerous or not. Further, the invention relates to a method of manufacturing such a needle.

### BACKGROUND OF THE INVENTION

In the field of oncology it is important to be able to discriminate tumor tissue from normal tissue. Golden standard is to inspect tissue at the pathology department after a biopsy or after surgical resection. A drawback of this current way of working is that real time feedback during the procedure of taking a biopsy or performing the surgical resection is missing. A way to provide feedback for instance in the case of a biopsy needle is to incorporate fibers to perform optical measurements at the tip of the needle. Various optical methods may be employed with diffuse reflectance spectroscopy (DRS) and autofluorescence measurement as the techniques that are most commonly investigated. Several probes are used to perform these measurements, but in general these probes have blunt end surfaces and are therefore not a direct integral part of the needle.

In U.S. Pat. No. 4,566,438 a sharp fiber-optic stylet is described in which two fibers are incorporated that could perform DRS and fluorescence measurements at the tip of the needle. However the fibers in the stylet are bevelled and as a result a significant part of the light in the fiber will undergo total internal reflection at the tip of the needle, reaching the cladding material of the fiber and then exiting the fiber. This travelling through the buffer may cause a significant amount of unwanted autofluorescence of the cladding material hampering the measurement of the tissue autofluorescence.

### SUMMARY OF THE INVENTION

With the attempt to at least alleviate the mentioned drawbacks, the following requirements are fulfilled by a needle according to an embodiment of the invention:
Needle must be sharp.
Integrating the fibers into the needle must not alter the penetration properties into the tissue.
Distance between excitation fiber end for fluorescence and the fluorescence detection fiber should be small.
The autofluorescence of the probe should be small compared to that generated in the tissue.
The shading effects of multilumen must be small.
The autofluorescence by the fiber itself must be small compared to the measured tissue signal.
The fibers in the needle may not extend beyond the bevel of the cannula.
The needle should be compatible with mass production.
The cost of the needle must be sufficiently low in order to make it disposable.
For correcting fluorescence signals for absorption and scattering, a DRS measurement has to be done with more than one fiber.

It might be an object of the invention to embed optical fibers into a needle such that the above requirements are fulfilled. It might be another object of the invention to provide a system for using the needle. A further object of the invention might be to provide a method for manufacturing such a needle.

These and other objects might be achieved by the subject matter according to the independent claims. Further embodiments of the present invention are described in the respective dependent claims.

To solve this problems a needle is proposed including a cannula or hollow shaft with a multilumen insert inside. The insert comprises at least two lumen. Both the insert as well as the cannula have bevelled ends. In the insert substantially straight cleaved fibers (i.e angle end face is small such that no total internal reflection at the interface may take place) are present that may be connected at the proximal end to a console. At least one of the distal fiber ends in the insert may protrude more than half the fiber diameter out of the insert. Furthermore, the bevel angle of the insert is different from the bevel angle of the cannula such that combination cannula and insert is such that the fiber ends do not protrude the bevel surface of the cannula.

In general, a needle according to an embodiment of the invention comprises a hollow shaft, an elongated insert and an optical fiber. The hollow shaft has a longitudinal axis and a first bevel formed at a distal end portion of the same and formed with an acute first angle to the longitudinal axis. The elongated insert has a second bevel formed at a distal end portion of the elongated insert and formed with an acute second angle to the longitudinal axis, wherein the first angle is smaller than the second angle. The optical fiber is arranged within the elongated insert and the elongated insert is arranged and fixed within the hollow shaft, so that the second bevel and the front surface of the fiber is located within the hollow shaft.

The tip of the needle, i.e. the bevel of the needle is in general slanted in order to allow easy entry into the tissue. Therefore, with ‘bevel’ is meant a geometrical structure allowing for introducing the needle into tissue. Usually, a shaft of a needle includes a circular cross section. The distal end of a needle shaft, in particular of a shaft of a hollow needle, is cut such that an oval surface is formed, which is inclined relative to the longitudinal axis of the shaft. Further, there is defined an angle between the longitudinal axis of the shaft and the inclined surface, i.e. the bevel. The bevel forms a pointed tip at the most distal end of the needle. Furthermore, the edge between the outer surface of the shaft and the inclined surface of the bevel might be sharpened.

In the following, geometrical aspects will be defined for a better understanding. First of all, the needle include a longitudinal main axis, usually the centre axis of a rotationally symmetrical shaft. Further, the tip portion of the shaft is cut at an angle to the main axis forming the bevel. Looking onto the surface of the bevel as well as on the shaft means looking from ‘above’. Accordingly, ‘under’ the needle is opposite to ‘above’. The pointed tip of the bevel is directed to the ‘front’ of the needle. As a result, looking from the ‘side’, it is possible to recognize the angle between the bevel and the main axis.

The wording ‘bevel’ might also include similar structures at the tip of the needle, which structures are useful for introducing the needle into a tissue. For example, the bevel might be a convex or concave surface, or the bevel might be a combination of several small surfaces, wherein these surfaces are connected to each other by steps or edges. It might also be possible that the cross section of the shaft is not completely cut by the bevel, such that an area remains which is blunt, i.e. is perpendicularly orientated relative to the longitudinal axis of the shaft. Such a ‘blunt’ end might include rounded edges or might also form a rounded leading edge. As another example, a sharp edge might be formed by two or more slanted surfaces being symmetrically or asymmetrically arranged to form the tip of the needle.

It should be noted that the bevel might form an acute angle with the shaft, such that the needle includes a pointed tip. Preferably, the acute angle might be approximately 20°.

According to another embodiment, the so called second bevel and the front surface of the fiber is located adjacent the so called first bevel within the hollow shaft.

The front surface of the fiber may be formed with a third angle to the longitudinal axis, wherein the third angle is greater than the second angle, wherein the third angle may be approximately a right angle to the longitudinal axis, and may be preferably a few degrees less than 90 degrees, i.e. between 80 degrees and 90 degrees.

The first bevel and the second bevel are orientated in the same direction, according to an embodiment of the invention.

The elongated insert of the needle may be removably fixed within the hollow shaft. That is, the insert may be fixed with its bevel in an appropriate relation to the bevel of the hollow shaft, during an insertion of the needle into tissue, and after said insertion, the insert may be released and pulled back out of the shaft, so that the needle may be used for an injection of a substance or a suction of for example a liquid out of a body.

According to a further embodiment of the invention, the elongated insert comprises two channels both with an open end at the second bevel of the elongated insert, wherein one open end is located more proximally than the other open end, and wherein the needle comprises two optical fibers each arranged within one of the channels, wherein the optical fiber which is arranged within the channels with the more proximally located open end may protrude out of the open end. The optical fiber may protrude more than half the diameter of the optical fiber out of the open end of the channel. With such an arrangement of fibers, with the end surfaces of the fibers in close proximity, especially fluorescence measurements are possible with increased signal.

According to another embodiment of the invention, the open ends of the two channels in the insert are located with a distance from each other which is greater than the diameter of the elongated insert. With such an arrangement of the fibers, especially diffuse reflectance spectroscopy is possible with good results.

For example, the distance is more than 1.1 times greater than the diameter. Particularly, the distance is more than 1.25 times greater than the diameter. Preferably, the distance is more than 1.5 times greater than the diameter. In other words, the distance between the fiber ends at the tip part of the needle should be as great as possible. It is noted that the distances are measured from the central axis of one of the fibers to the central axis of the other one of the fibers.

According to another aspect, the shaft and tip of the needle might be made of metal, wherein the metal might be MRI compatible such as Titanium. The needle tip might also be made of a ceramic material. This has the advantage of being mouldable in various shapes while still allowing for a sharp and robust needle tip. On the other end, the holder part might be made by plastic injection moulding. The elongated insert may be made of a plastic material and may be coated with a metal coating or a coating having low autofluorescence.

According to a further embodiment of the invention, the hollow shaft of the needle further includes facets formed at both sides of the bevel.

A ‘facet’ may by a small and plane surface. Usually, a ‘facet’ may be realized by cutting away a small area of a body thereby achieving a surface with edges to other surfaces of the body. The contour of a facet may be affected by the angle of cutting. Furthermore, the surface of a facet may be convex or concave, i.e. the facet may be curved forming a part-cylindrical shape. The edges of the facet may preferably be sharpened or may be rounded and thus blunt.

Principally, it is possible to introduce a needle or instrument into tissue by cutting the tissue or displacing the tissue. Accordingly, the edges of a needle or instrument will be sharp or blunt. It will be understood that a combination of cutting and displacing or squeesing the tissue is also possible. Depending from the application, the needle or instrument will more or less cut and/or displace.

According to another embodiment of the invention, the elongated insert comprises three channels each with an open end at the second bevel, wherein a first open end is located distally, a second open end is located in the proximity of the first open end, and a third open end is located proximally, and wherein the needle comprises three optical fibers arranged in the three channels of the elongated insert, respectively. Such an arrangement of the fibers allows for a combination of diffuse reflectance spectroscopy and fluorescence measurments.

To further enhance the functionality of the needle, the channel with the first open end is formed as a pair of channels located side by side distally at the second bevel of the elongated insert, so that four channels with fibers are integrated into the needle. It should be noted that instead of the most distally arranged channel, also the channel with the second open end may be a pair of channels arranged side by side.

In accordance with another aspect of the invention, a system for tissue inspection comprises a needle as described above together with a console including a light source, a light detector and a processing unit for processing the signals provided by the light detector, wherein one of the light source and light detector may provide wavelength selectivity. The light source may be one of a laser, a light-emitting diode or a filtered light source, and the console may further comprise one of a fiber switch, a beam splitter or a dichroic beam combiner.

According to an embodiment of the invention, the system is adapted to perform at least one out of the group consisting of diffuse reflectance spectroscopy, fluorescence spectroscopy, diffuse optical tomography, differential path length spectroscopy, and Raman spectroscopy.

According to a further aspect of the invention, a method for producing a needle as described above comprises the steps of manufacturing a hollow shaft including forming a first bevel with an acute first angle to a longitudinal axis of the hollow shaft, manufacturing an elongated insert including forming a second bevel with an acute second angle to the longitudinal axis and forming at least one channel for accommodating an optical fiber, wherein the second angle is greater than the first angle, positioning and fixing at least one fiber in a respective channel, positioning and fixing the elongated insert within the hollow shaft, so that the second bevel and the front surface of the at least one fiber is located within the hollow shaft. The at least one fiber may be positioned within the at least one channel, so that a pocket is formed at the open end of the channel at the second bevel of the elongated insert.

The invention might also be related to a computer program for the processing unit of the system according to the invention. The computer program is preferably loaded into a working memory of a data processor. However, the computer program may also be presented over a network like the worldwide web and may be downloaded into the working memory of a data processor from such a network. The computer program might control the emitting of light, might process the signals coming from the light detector at the proximal end of the detector fiber(s). These data might then be visualized on a monitor.

It has to be noted that embodiments of the invention are described with reference to different subject matters. In particular, some embodiments are described with reference to method steps whereas other embodiments are described with reference to devices or systems. However, a person skilled in the art will gather from the above and the following description that, unless other notified, in addition to any combination of features belonging to one type of subject matter also any combination between features relating to different subject matters is considered to be disclosed with this application.

The aspects defined above and further aspects, features and advantages of the present invention may also be derived from the examples of embodiments to be described hereinafter and are explained with reference to examples of embodiments. The invention will be described in more detail hereinafter with reference to examples of embodiments but to which the invention is not limited.

### BRIEF DESCRIPTION OF THE DRAWINGS

FIG. 1 shows a distal tip portion of a first embodiment of a needle according to the invention.

FIG. 2 shows a distal tip portion of a second embodiment of a needle according to the invention.

FIG. 3 shows results of fluorescence measurements with emitting and receiving fibers at different positions.

FIG. 4 shows results of fluorescence measurements with one or more fibers protruding different amounts out of the channel.

FIG. 5 shows a system according to the invention.

FIG. 6 shows the absorption of different biological chromophores.

FIG. 7 is a flow chart illustrating a method according to the invention.

The illustration in the drawings is schematically only and not to scale. It is noted that similar elements are provided with the same reference signs in different figures, if appropriate.

### DETAILED DESCRIPTION OF EMBODIMENTS

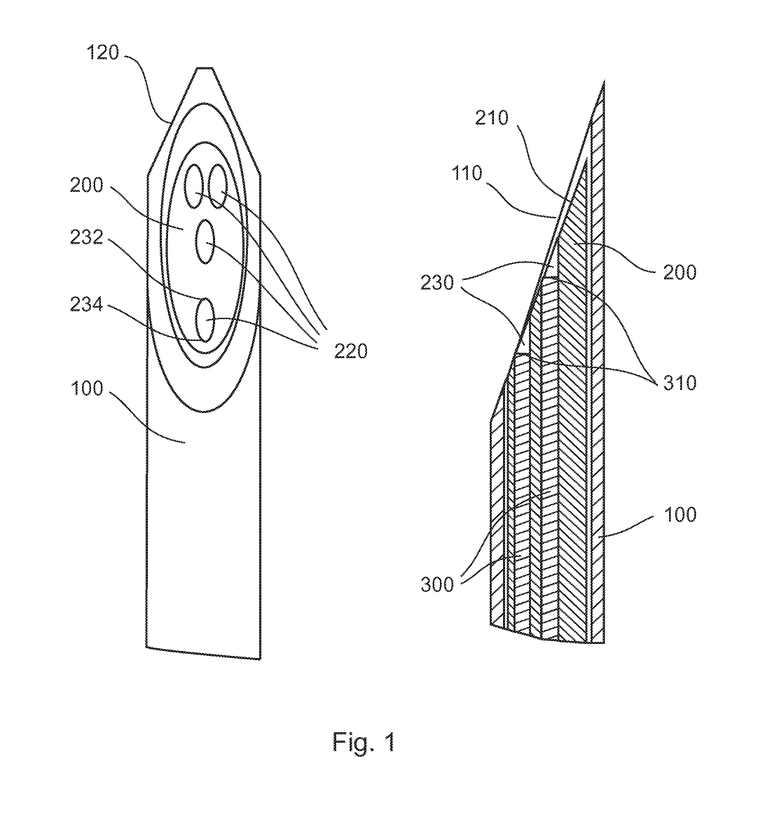

FIG. 1 illustrates a distal tip portion of a needle according to a first embodiment of the invention. The needle comprises a shaft 100 and an elongated insert 200. The shaft 100 is formed with a first bevel 110 and the insert 200 is formed with a second bevel 210, with both bevels orientated in the same direction. As can be seen in FIG. 1, the first bevel 110 is formed with an angle which is different to the angle of the second bevel 210.

Furthermore, the shaft 100 has facets 120 formed at the sides of the bevel so that the facets are orientated to the front as well as the side of the tip. The insert 200 further includes channels 220 having open ends at the surface of the bevel 210. Due to the angled bevel, the open ends of the channels 220 provide pockets 230 with a distal end 232 of the pocket 230 and a proximal end 234. Within the channels 220, optical fibers 300 with front surfaces 310 are arranged.

It is noted that the right view of FIG. 1 is a section view along the center line of the left view, so that only two of the four channels are visible in the section view.

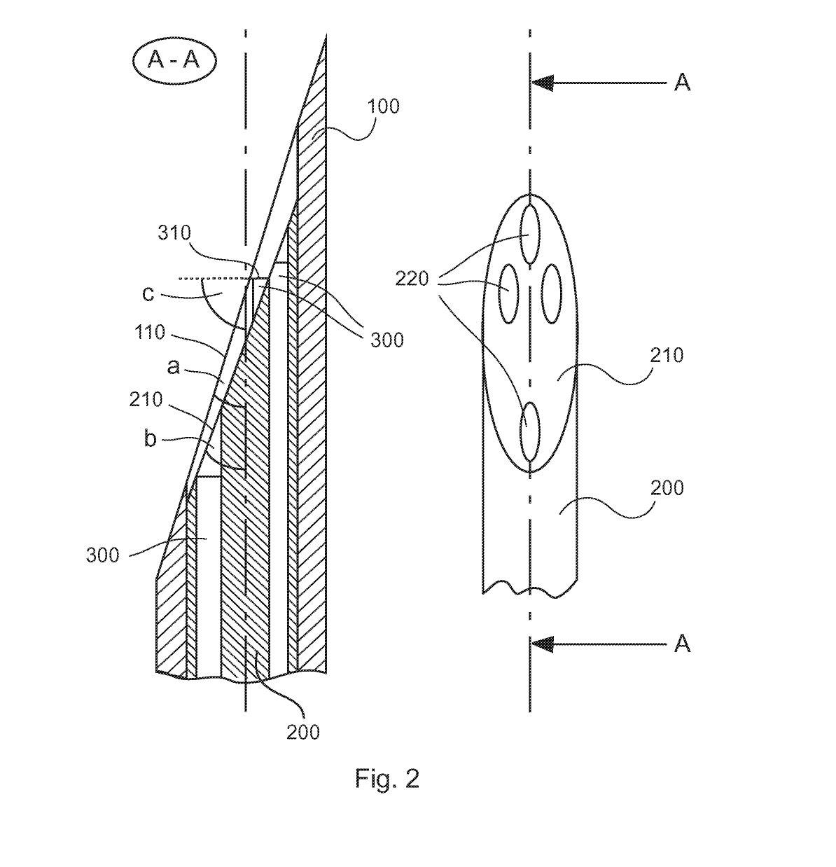

FIG. 2 shows a second embodiment of a needle according to the invention, wherein the right view shows only the distal tip portion of the elongated insert 200 and the left view is a section view along the center line of said insert, but together with the shaft 100 of the needle. In this embodiment, a pair of channels 220 is formed at the bevel 210 between the most distally arranged channel and the middle of the cross section of the insert. Accordingly, the tip of an optical fiber 300 is protruding beyond the surface of the second bevel 210. The optical fiber 300 does not protrude beyond the first bevel 110 of the shaft.

Further, different angles are shown in FIG. 2. Between the longitudinal axis of the needle and the first bevel 110 of the shaft 100, a first angle ‘a’ is formed which is an acute angle. Between the longitudinal axis and the second bevel 210 of the insert 200, a second angle ‘b’ is formed which is also an acute angle but which is greater than the first angle ‘a’. Furthermore, the front surface 310 of the optical fiber may be formed with a third angle ‘c’ which is preferrably less but near 90 degrees.

To manufacture such a needle with a multilumen insert, a cannula is used. The insert is typically made of plastic material with well defined lumen at positions that define the distances between the fibers that may be inserted in these lumen. The fibers used in the lumen are typically straight cut or only a moderate angle in such a way that (partly) total internal reflection at the fiber end is prevented. When total internal reflection occurs light reflected at the fiber end will end up in the cladding of the fiber. Depending on what material surrounds the fiber, part of this light will be reflected back into the core of the fiber and is able to leave the fiber. For diffuse reflectance this is less of a problem but for fluorescence it causes a significant amount of background fluorescence. This hampers the investigation of the fluorescence generated by the tissue.

The insert at the distal end is bevelled by an angle that is greater than the angle of the bevel of the cannula. In this way when assembling the fibers inside the multilumen they may protrude somewhat beyond the bevel of the insert without protruding beyond the bevel of the cannula. This is important in order not to affect the insertion properties of the needle in the tissue.

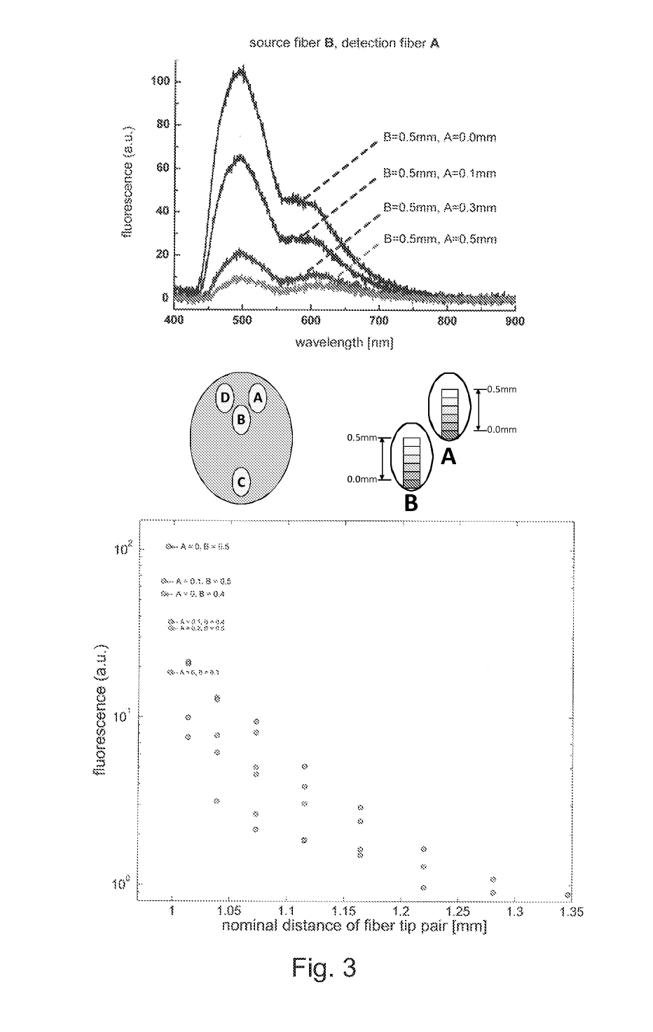

The simplest way to assemble the fiber in the insert is by positioning the fiber end equal to the start of the pocket as depict in FIG. 1. For diffuse reflectance this is a possible option but for fluorescence this is not preferred. For fluorescence detection the distance between the source and the detection fiber ends should be small to have optimal signal. This can be seen from the measurements shown in FIG. 3. As depict in the middle of FIG. 3, fibers are located in pockets A and B which are shiftly arranged on the bevel. For the measurements, the fibers ends are at different positions in the pockets and thus relative to the bevel surface.

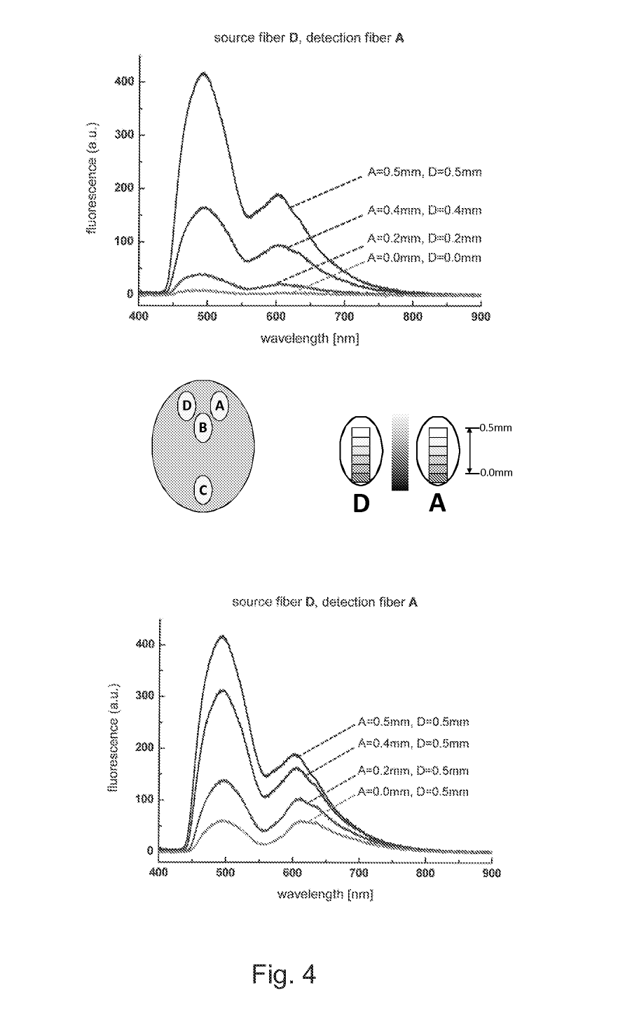

A further observation can be made when the two pockets, i.e. pockets A and D, are arranged side by side and adjacent to each other as is the case in FIG. 4. When the fibers are substantially equal to the start of the pocket the shading effect of the walls of the pockets is significant leading to smaller signals. This it schematically visualized by the bar between the pockets A and D in the middle of FIG. 4. So in this case although the distance between the fiber does not changes when they both protrude the same amount beyond the start of the pocket, the signal becomes higher when they protrude more because of the reduced effect of the side wall of the pocket. Therefore, in case of fluorescence at least one of the fiber ends should protrude beyond the start of the pocket. Advantageously, the fiber end protrudes more than half of the diameter of the fiber beyond the start of the pocket. In a further embodiment, the fiber end protrudes more than the diameter of the fiber beyond the start of the pocket.

The insert may be produced in mass production. Producing straight cut fibers may be done in batches. Assembling fibers in the multilumen may be well controlled making these needles compatible with mass production. Furthermore, because of this way of assembling, a rather low cost needle may be assured.

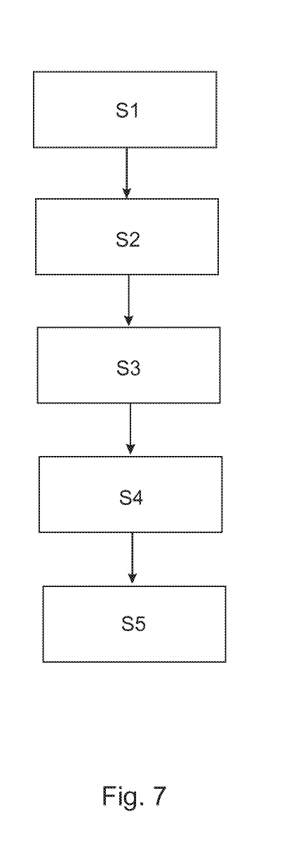

FIG. 7 is a flow chart, showing the steps of a method for producing a needle according to the invention. It will be understood, that the steps described with respect to the method, are major steps, wherein these major steps might be differentiated or divided into several sub steps. Furthermore, there might be also sub steps between these major steps. Therefore, a sub step is only mentioned, if said step is important for the understanding of the principles of the method according to the invention.

In step S1, a hollow shaft is manufactured, wherein this step includes the forming of a first bevel with an acute first angle to a longitudinal axis of the hollow shaft.

In step S2, an elongated insert is manufactured, wherein this step includes the forming of a second bevel with an acute second angle to the longitudinal axis and the forming of at least one channel for accommodating an optical fiber, wherein the second angle is greater than the first angle.

In step S3, at least one fiber is positioned and fixed in a channel. The at least one fiber may be positioned within the at least one channel, so that a pocket is formed at the open end of the channel at the second bevel of the elongated insert.

In step S4, the elongated insert is positioned within the hollow shaft, so that the second bevel and the front surface of the at least one fiber is located within the hollow shaft, i.e. so that the front surface of the fiber does not protrude beyond the surface of the first bevel of the shaft.

In step S5, the elongated insert is removably fixed relative to the shaft. Preferably, this is achieved by a releasable connection between the insert and the shaft at the holder part of the needle.

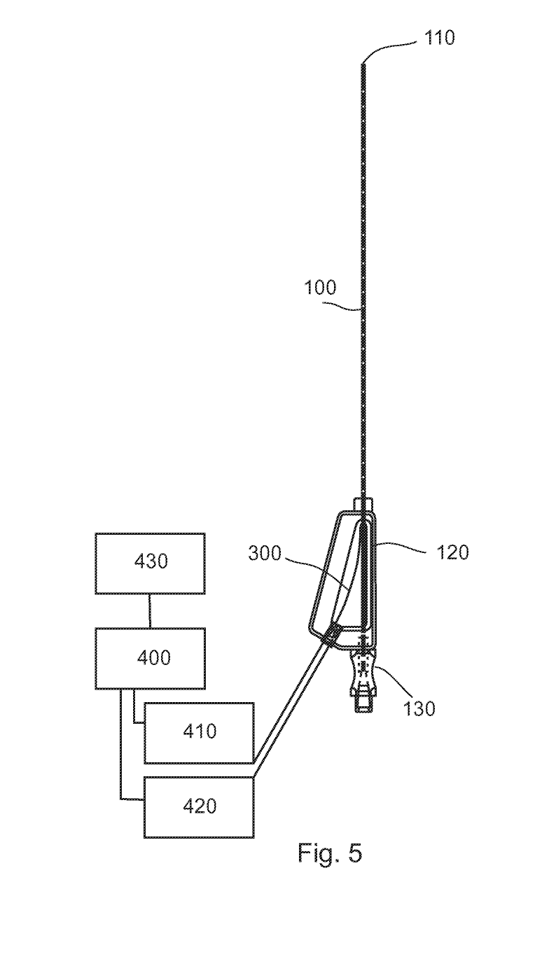

As illustrated in FIG. 5, the needle with shaft 100 and insert including fibers 300 may be connected to an optical console. The optical console contains a light source 410 enabling light to be provided via one or more of the fibers 300 to bevel 110 at the distal end of the needle. The scattered light is collected by one or more other fibers 300 and is guided towards the detector 420 or detectors. The amount of reflected light measured at the “detection” fiber, is determined by the absorption and scattering properties of the probed structure (e.g. tissue). The data may be processed by a processing unit 400 using a dedicated algorithm. For diffuse reflectance measurements, either the light source or the detector or a combination of both must provide wavelength selectivity. For instance, light can be coupled out of the distal tip through at least one fiber, which serves as a source, and the wavelength is swept from e.g. 500-1600 nm, while the light detected by at least one detection fiber is sent to a broadband detector. Alternatively, broadband light may be provided by at least one source fiber, while the light detected by at least one detection fiber is sent to a wavelength-selective detector, e.g. a spectrometer.

For a detailed discussion on diffuse reflectance measurements see R. Nachabe, B. H. W. Hendriks, A. E. Desjardins, M. van der Voort, M. B. van der Mark, and H. J. C. M. Sterenborg, “Estimation of lipid and water concentrations in scattering media with diffuse optical spectroscopy from 900 to 1600 nm”, J. Biomed. Opt. 15, 037015 (2010).

For fluorescence measurements the console must be capable of providing excitation light to at least one source fiber while detecting tissue-generated fluorescence through one or more detection fibers. The excitation light source may be a laser (e.g. a semiconductor laser), a light-emitting diode (LED) or a filtered light source, such as a filtered mercury lamp. In general, the wavelengths emitted by the excitation light source are shorter than the range of wavelengths of the fluorescence that is to be detected. It is preferable to filter out the excitation light using a detection filter in order to avoid possible overload of the detector by the excitation light. A wavelength-selective detector, e.g. a spectrometer, is required when multiple fluorescent entities are present that need to be distinguished from each other.

In case fluorescence measurements are to be combined with diffuse reflectance measurements, the excitation light for measuring fluorescence may be provided to the same source fiber as the light for diffuse reflectance. This may be accomplished by, e.g., using a fiber switch, or a beam splitter or dichroic beam combiner with focusing optics. Alternatively, separate fibers may be used for providing fluorescence excitation light and light for diffuse reflectance measurements.

Although diffuse reflectance spectroscopy is described above to extract tissue properties also other optical methods may be envisioned like diffuse optical tomography by employing a plurality of optical fibers, differential path length spectroscopy, Raman spectroscopy. Furthermore, the system may also be employed when contrast agents are used instead of only looking at autofluorescence.

In accordance with the invention the following algorithm may be utilized to derive optical tissue properties such as the scattering coefficient and absorption coefficient of different tissue chromophores: e.g. hemoglobin, oxygenated haemoglobin, water, fat etc. These properties are different between normal healthy tissue and diseased (cancerous) tissue.

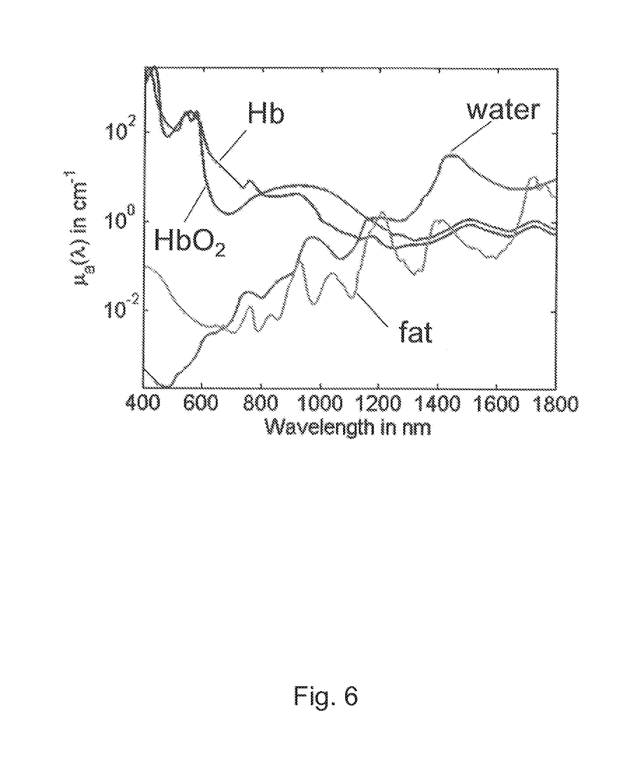

The main absorbing constituents in normal tissue dominating the absorption in the visible and near-infrared range are blood (i.e. hemoglobin), water and fat. In FIG. 6 the absorption coefficient of these chromophores as a function of the wavelength are presented. Note that blood dominates the absorption in the visible range, while water and fat dominate in the near infrared range.

The total absorption coefficient is a linear combination of the absorption coefficients of for instance blood, water and fat (hence for each component the value of that shown in FIG. 6 multiplied by its volume fraction). By fitting the model to the measurement while using the power law for scattering (see R. Nachabe, B. H. W. Hendriks, A. E. Desjardins, M. van der Voort, M. B. van der Mark, and H. J. C. M. Sterenborg, “Estimation of lipid and water concentrations in scattering media with diffuse optical spectroscopy from 900 to 1600 nm”, J. Biomed. Opt. 15, 037015 (2010)) we may determine the volume fractions of the blood, water and fat as well as the scattering coefficient. With this method we may now translate the measured spectra in physiological parameters that may be used to discriminate different tissues.

Another way to discriminate differences in spectra is by making use of a principal components analysis. This method allows classification of differences in spectra and thus allows discrimination between tissues. It is also possible to extract features from the spectra.

How to extract the intrinsic fluorescence from the measured fluorescence may be found for instance in Zhang et al., Optics Letters 25 (2000) p 1451.

The needles according to the invention may be used in minimally invasive needle interventions such as low-back pain interventions or taking biopsies in the field of cancer diagnosis or in case where tissue characterization around the needle is required.

While the invention has been illustrated and described in detail in the drawings and foregoing description, such illustration and description are to be considered illustrative or exemplary and not restrictive; the invention is not limited to the disclosed embodiments. Other variations to the disclosed embodiments may be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

### LIST OF REFERENCE SIGNS

100 hollow shaft
110 first bevel
120 holder part
130 connector
200 elongated insert
210 second bevel
220 channel
230 pocket
232 distal end of pocket
234 proximal end of pocket
300 optical fiber
310 front surface
400 processing unit
410 light source
420 light detector
430 monitor

1. A needle comprising:
a hollow shaft having a longitudinal axis and a first bevel formed at a distal end portion of the hollow shaft and with an acute first angle (a) to the longitudinal axis,
an elongated insert having a second bevel formed at a distal end portion of the elongated insert and with an acute second angle (b) to the longitudinal axis,
wherein the first angle (a) is smaller than the second angle (b), and
an optical fiber, wherein the optical fiber is arranged within the elongated insert and the optical fiber protrudes beyond the surface of the second bevel,
wherein the elongated insert is arranged and fixed within the hollow shaft, so that the second bevel and a front surface of the fiber is located within the hollow shaft.

2. The needle of claim 1, wherein the front surface of the optical fiber is formed with a third angle (c) to the longitudinal axis, wherein the third angle is greater than the second angle (b).

3. The needle of claim 1, wherein the elongated insert is removably fixed within the hollow shaft.

4. The needle of claim 1, wherein the elongated insert comprises two channels both with an open end at the second bevel of the elongated insert, wherein one open end is located more proximally than the other open end, and wherein the needle comprises two optical fibers each arranged within one of the channels.

5. The needle of claim 4, wherein the optical fiber which is arranged within the channel with the more proximally located open end protrudes out of the open end.

6. The needle of claim 4, wherein the open ends of the two channels are located with a distance from each other which is greater than the diameter of the elongated insert.

7. The needle of claim 1, wherein the elongated insert is coated with a metal coating or a coating having low autofluroescence.

8. The needle of claim 1, wherein the elongated insert comprises three channels each with an open end at the second bevel, wherein a first open end is located distally, a second open end is located in the proximity of the first open end, and a third open end is located proximally, and wherein the needle comprises three optical fibers arranged in the three channels of the elongated insert, respectively.

9. A system for tissue inspection, comprising:
a needle according to claim 1, and
a console including a light source, a light detector and a processing unit for processing the signals provided by the light detector.

10. The system of claim 9, wherein one of the light source and light detector provides wavelength selectivity.

11. The system of claim 9, wherein the light source is one of a laser, a light-emitting diode or a filtered light source.

12. The system of claim 9, wherein the console further comprises one of a fiber switch, a beam splitter or a dichroic beam combiner.

13. The system of claim 9, wherein the system is adapted to perform at least one out of the group consisting of diffuse reflectance spectroscopy, diffuse optical tomography, differential path length spectroscopy, and Raman spectroscopy.

14. A method for producing a needle according to claim 1, the method comprising the steps of:
manufacturing a hollow shaft including forming a first bevel with an acute first angle (a) to a longitudinal axis of the hollow shaft,
manufacturing an elongated insert including forming a second bevel with an acute second angle (b) to the longitudinal axis and forming at least one channel with an open end at the second bevel, wherein the second angle (b) is greater than the first angle (a),
positioning and fixing at least one optical fiber in a respective channel, so that the at least one optical fiber protrudes beyond the surface of the second bevel,
positioning and fixing the elongated insert within the hollow shaft, so that the second bevel and a front surface of the at least one optical fiber is located within the hollow shaft.

15. The method of claim 14, wherein the at least one optical fiber is positioned within the channel the open end of which is located more proximally at the second bevel, so that the optical fiber protrudes more than half the diameter of the optical fiber out of the open end.
